(19) United States
(12) Patent Application Publication
(10) Pub. No.: US 2020/0353279 A1
(43) Pub. Date: Nov. 12, 2020

(54) Corneal Crosslinking With Catalyst Distribution Control

(71) Applicant: TECLens, LLC, St. James, NY (US)

(72) Inventors: Edward Paul Harhen, Duxbury, MA (US); Patrick David Lopath, Stamford, CT (US)

(73) Assignee: TECLens, LLC, St. James, NY (US)

(21) Appl. No.: 16/830,641

(22) Filed: Mar. 26, 2020

Related U.S. Application Data
(60) Provisional application No. 62/825,388, filed on Mar. 28, 2019.

(51) Int. Cl. A61N 5/06; A61F 9/007; A61K 47/02; A61F 9/00; A61K 47/22

(52) U.S. Cl. CPC A61N 5/062; A61F 9/007; A61N 2005/0661; A61F 9/0017; A61K 47/22; A61K 47/02

(57) ABSTRACT

In corneal crosslinking, the anterior surface of the cornea of the eye is maintained in contact with a first liquid having a first concentration of a crosslinking catalyst such as riboflavin, so that the catalyst enters the cornea and forms a first concentration profile (t1) in the corneal stroma. The anterior surface of the cornea is then maintained in contact with one or more additional liquids having concentration of the catalyst lower than the first concentration so that the catalyst forms a second concentration profile (t4, t5, t6) in the stroma. In the second concentration profile, the maximum concentration of the catalyst desirably is posterior to the anterior surface of the cornea. The cornea is irradiated and crosslinked. The second concentration profile facilitates crosslinking deep within the stroma.

### CROSS-REFERENCE TO RELATED APPLICATIONS

The present application claims the benefit of U.S. Provisional Patent Application No. 62/825,388, filed Mar. 28, 2019, the disclosure of which is incorporated herein by reference herein.

### BACKGROUND OF THE INVENTION

The cornea of the human eye has an outer (anterior) epithelial layer, an inner (posterior) endothelium and a relatively thick stroma positioned between the epithelium and endothelium. A thin, smooth membrane, known as Bowman's Layer, lies between the anterior epithelial layer and the anterior surface of the stroma. Another thin membrane, known as Descemet's membrane, lies between the posterior surface of the stroma and the posterior endothelium. The stroma, as well as Bowman's Layer, contains strong collagen fibers which define the shape of the cornea.

Corneal crosslinking (also referred to as “CXL”) typically is performed by introducing a photoactive catalyst, most commonly riboflavin, into the stroma and applying light, most commonly long wavelength ultraviolet light (“UV”), to the cornea. Exposure to the light in presence of the catalyst causes crosslinking of the collagen in the stroma which strengthens the stroma. This strengthening causes reshaping of the cornea. Corneal crosslinking can be used to treat keratoconus and refractive errors such as hyperopia and myopia. A goal in cornea crosslinking is to cause strengthening of the stroma throughout its thickness.

The structure of the eye limits the ability to induce an elevated riboflavin concentration in the stroma of the cornea. The tight junctions of the epithelium are significant barriers for chemical transport of large molecules between the bolus of solution that sits on the epithelium and the stroma.

Ultraviolet light is attenuated by riboflavin. Some photobleaching of the riboflavin occurs when riboflavin is exposed to ultraviolet. The photochemistry of the riboflavin/UV interaction is complex, with pathways for photodegradation of the riboflavin, as well as recombination into a photoactive state again, thus the rate of photobleaching is dependent on the riboflavin concentration, the intensity of the UV light and the duration of illumination. Diffusion according to Fick's Law can be used to describe the time dependent concentration gradient of riboflavin across the stroma.

During crosslinking, it is important that most of the incident UV light is attenuated before it reaches the endothelium on the posterior side of the cornea, so as to limit exposure of the endothelium and the natural lens to ultraviolet. Riboflavin's ability to attenuate UV is dependent on the concentration in the tissue and the thickness of the riboflavin-loaded tissue layer. Attenuation increases with increasing riboflavin concentrations.

The epithelium is approximately 50 μm thick and the endothelium is about 15 μm thick. The normal healthy cornea is about 500-600 microns thick in total, i.e., the stroma is on the order of 450-550 μm thick.

In conventional corneal crosslinking procedures, riboflavin is introduced into the cornea by applying a bolus of a riboflavin solution to the anterior surface of the cornea. When a bolus of riboflavin solution is applied to an untreated, intact epithelium, very little of the riboflavin traverses the epithelium because the tight junctions between the epithelial cells form a barrier to large molecule of riboflavin. Stated another way, an untreated, intact epithelium forms a barrier to diffusion of riboflavin at the anterior surface of the cornea. In order to facilitate riboflavin uptake in the corneal stroma as a precursor to ultraviolet light mediated corneal crosslinking (CXL), the epithelial barrier must be defeated in some manner. The original CXL protocol (the Dresden protocol) called for the removal of the epithelium. While this achieves the desired access route to the stroma for the photoactive riboflavin, it is very painful for the patient and increases the risk of post-operative infection. More recent CXL protocols have tried less invasive ways to defeat the barrier, including iontophoresis, ultrasonic permeation, and drugs which disable the barrier function. The precise method used to defeat the barrier is not relevant here, as the disclosed invention can be applied with any technique.

Commercially available riboflavin formulations are typically 0.1 to 0.25% (w/w) riboflavin and are applied for 30 minutes after the epithelial barrier has been defeated in some manner. With the barrier eliminated, riboflavin can easily enter the stroma and reach a predictable concentration gradient that can then be exploited to achieve deep crosslinking by application of an appropriate UV dose.

As a convention, the present description of the invention will use the term ‘anterior surface of the cornea’ to mean the most anterior surface remaining after corneal preparation. If the anterior epithelium is removed, as in the Dresden protocol, the anterior surface of the cornea will mean the exposed anterior surface of the stroma or Bowman's membrane. If the anterior epithelium remains in place as, for example, where a treatment which disables the barrier without removing the anterior epithelium is used, the anterior surface of the cornea will mean the most anterior remaining layer of the epithelium. Either way, it is assumed herein, unless otherwise stated, that the barrier function of the epithelium has been compromised in some manner to allow passage of riboflavin through.

In most CXL protocols in use today in the clinic, riboflavin is reapplied to the anterior surface of the cornea at numerous intervals during the treatment with UV. Improvement in this technique to encourage deep crosslinking would be desirable.

### BRIEF SUMMARY OF THE INVENTION

One aspect of the invention provides methods of corneal crosslinking. The method according to this aspect of the invention desirably includes the step of maintaining the anterior surface of the cornea of an eye of a living subject in contact with a first liquid having a first concentration of a crosslinking catalyst dissolved therein so that the catalyst penetrates into the cornea and forms a first concentration profile within the stroma of the cornea. After this step, the method desirably further includes the step of maintaining the anterior surface of the cornea in contact with one or more additional liquids having concentrations of the catalyst lower than the first concentration so that catalyst within the cornea forms a second concentration profile within the stroma different from the first concentration profile. The method desirably further includes the step of irradiating the cornea with light so that the light, in conjunction with the catalyst, causes crosslinking of collagen in the stroma. The step of maintaining the cornea in contact with the additional liquids desirably is performed before the irradiating step, concomitantly with the irradiating step, or both before and concomitantly with the irradiating step.

The second concentration profile desirably has a lower concentration of the catalyst in an anterior portion of stroma of the cornea than the first concentration profile Desirably, in the second concentration profile concentration of the catalyst in an anterior portion of the stroma is less than or equal to a maximum concentration of the catalyst in the stroma and the maximum concentration of the catalyst in the stroma is disposed at a location posterior to the anterior portion of the stroma. Stated another way, in the second concentration profile, the concentration of catalyst desirably increases with depth from the anterior surface of the cornea through at least a part of the cornea adjacent the anterior surface. This promotes crosslinking deep within the corneal stroma. The steps of applying the first and second liquids may be repeated at intervals during the irradiating step.

As further explained below, the first concentration of the catalyst in the first liquid sets the boundary condition at the anterior surface of the cornea for diffusion of the catalyst into the cornea. Diffusion under this boundary condition forms the first concentration profile. The second, lower concentration of catalyst in the second liquid sets a different boundary condition at the anterior surface of the cornea.

A further aspect of the invention provides apparatus which can be used to treat the eye of a living subject with a catalyst for corneal crosslinking. Apparatus according to this aspect of the invention desirably includes at least one reservoir adapted to rest on the eye of the subject and to define a space in alignment with the cornea of the eye. The apparatus may further include a plurality of liquid sources holding a plurality of liquids having different concentrations of a crosslinking catalyst such as riboflavin, at least one of the concentrations of the catalyst being greater than zero. The apparatus desirably includes a means for supplying one or more of the liquids to the space so as to provide liquid in the space and vary the concentration of the catalyst in liquid which is disposed in the space with time and thereby contact the cornea with a succession of liquids including a first liquid having a first concentration of catalyst and one or more additional liquids having concentrations of catalyst lower than the first concentration.

The means for supplying may include means for blending liquids from a plurality of the sources and supplying the blended liquid to the space. The means for supplying may be operative to vary the proportions of liquids from different sources included in the blended liquid so as to vary a concentration of catalyst in the blended liquid.

Stated another way, apparatus for treating the eye of a living subject with a catalyst for corneal crosslinking may include means for maintaining the anterior surface of the cornea of an eye of a living subject in contact with a first liquid having a first concentration of a crosslinking catalyst dissolved therein so that the catalyst penetrates into the cornea and forms a first concentration profile within the stroma of the cornea. This apparatus may include means for maintaining the anterior surface of the cornea in contact with one or more additional liquids having concentrations of the catalyst lower than the first concentration after the first concentration profile has been formed so that catalyst within the cornea forms a second concentration profile within the stroma different from the first concentration profile. The apparatus optionally may also include means for irradiating the cornea with light so that the light, in conjunction with the catalyst, causes crosslinking of collagen in the stroma.

Desirably, the means for maintaining the anterior surface of the cornea in contact with the more additional liquids is operative to maintain the anterior surface of the cornea in contact with the one or more additional liquids so that, in the second concentration profile, a concentration of the catalyst in an anterior portion of the stroma is less than a maximum concentration of the catalyst in the stroma and the maximum concentration of the catalyst in the stroma is disposed at a location posterior to the anterior portion of the stroma. Desirably, the means for maintaining the anterior surface of the cornea in contact with the more additional liquids is operative to maintain the anterior surface of the cornea in contact with the one or more additional liquids so that, in the second concentration profile, the maximum concentration of the catalyst in the stroma occurs at a depth from the anterior surface of the corneal stroma equal to at least 5 percent of the thickness of the corneal stroma. The means for irradiating desirably is operative to begin irradiation of the cornea after the means for maintaining the anterior surface of the cornea in contact with the more additional liquids has begun such contact, and desirably after contact with the more additional liquids has terminated.

The apparatus may further include means for restoring a barrier to diffusion of the catalyst at the anterior surface of the cornea as, for example, by a applying an a barrier liquid to the anterior surface of the cornea, the catalyst being substantially insoluble in the barrier liquid. Most preferably, the catalyst is substantially insoluble in the barrier liquid, and the barrier liquid is transparent to the light applied to irradiate the cornea and cause crosslinking. The barrier liquid may remain in contact with the cornea during irradiation. The apparatus desirably is arranged to supply the barrier liquid with oxygen dissolved therein, so that the barrier liquid serves to supply oxygen to the cornea.

A further aspect of the invention, which is usable independently of the other aspects of the invention pertaining to the catalyst concentration profile, provides apparatus for corneal crosslinking. The apparatus according to this aspect of the invention desirably includes a structure having an interior surface adapted to overlie a surface of the cornea of an eye of a mammalian subject; a source of light; a liquid supply constructed and arranged to supply a liquid having oxygen dissolved therein to a space between the cornea and the structure while the structure is overlying the cornea and while light is passing from the light source to the cornea so that the liquid passes out of the space, and means for returning the liquid passing out of the space to the liquid supply. Desirably, the structure is adapted to direct light from the light source into the cornea. Desirably, the structure has a form corresponding to the form of a contact lens.

### BRIEF DESCRIPTION OF THE DRAWINGS

FIG. 1 is a graph schematically depicting a catalyst concentration profile used in a conventional corneal crosslinking procedure.

FIG. 2 is a graph depicting catalyst concentration profiles at various times during a method according to one embodiment of the invention.

FIG. 3 is a schematic view depicting apparatus according to an embodiment of the invention.

FIG. 4 is a schematic view depicting apparatus according to a further embodiment of the invention.

FIG. 5 is a fragmentary schematic view depicting a component usable in apparatus according to certain embodiments of the invention.

FIG. 6 is a schematic view depicting apparatus according to a further embodiment of the invention.

### DETAILED DESCRIPTION

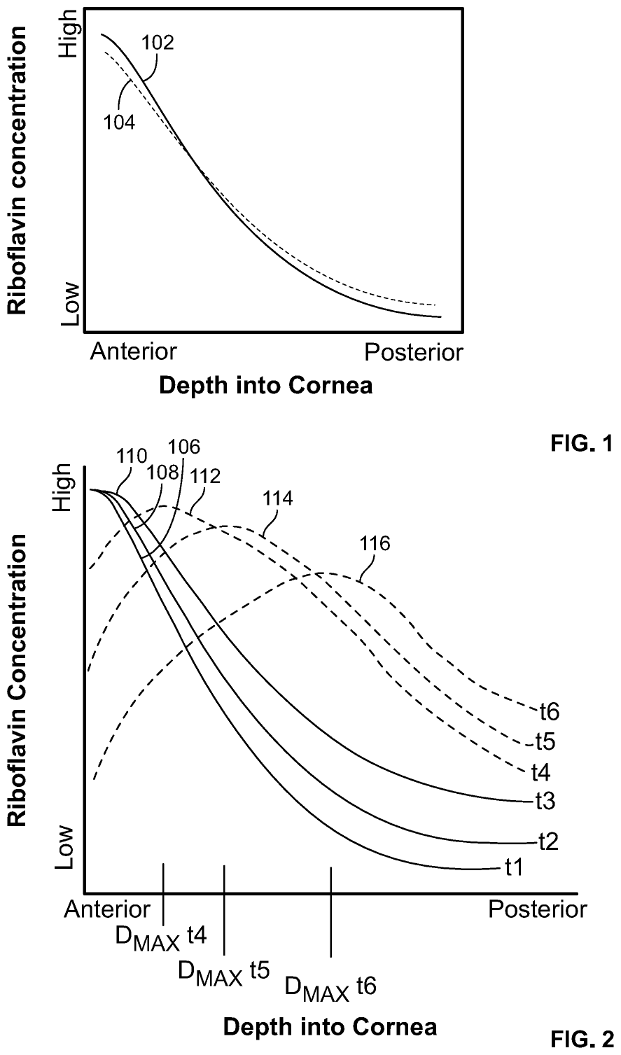

One aspect of the present disclosure incorporates the realization that the riboflavin concentration profile achieved with the conventional procedure has significant drawbacks. A typical concentration profile achieved in the conventional procedure is shown by curve 102 in FIG. 1. The term “concentration profile” as used herein refers to the distribution of the catalyst versus depth from the anterior surface of the cornea at a given time. The concentration profile depicted in curve 102 is a profile which exists immediately after a cornea which is initially devoid of catalyst has been contacted with a single liquid having a fixed concentration of the catalyst. Diffusion of the catalyst into the cornea during the contact is governed by Fick's law of diffusion, with a boundary condition that at the anterior surface of the cornea, the catalyst concentration is equal to the fixed concentration of the catalyst in the solution.

In the concentration profile shown by curve 102, the maximum catalyst concentration is at the anterior surface of the stroma, and the catalyst concentration decreases monotonically in the posterior direction. In the conventional procedure, no deliberate steps are taken to modify the concentration profile before UV irradiation begins. Because the catalyst continues to diffuse within the cornea after the liquid contact ceases, the concentration profile will change with time. Thus, catalyst diffuses from the high-concentration anterior region towards the lower-concentration posterior region. This results in concentration profile as indicated by curve 104 in FIG. 1. This concentration profile has a somewhat lower peak, but still has a maximum concentration at the anterior surface of the cornea. In the conventional procedure, irradiation of the cornea with UV light begins while the concentration profile is as shown by curve 102 or 104, or at some state intermediate between these. This type of riboflavin concentration profile, with a high concentration anteriorly, rapidly attenuates the UV light impinging on the anterior surface, so that minimal crosslinking occurs in the posterior region of stroma. In the conventional procedure, the irradiation may be interrupted and the catalyst may be reapplied. The concentration profile after reapplication may be more complex, but again will have a high concentration of the catalyst in the anterior region and a catalyst concentration which decreases with increasing distance from the anterior surface, at least in the anterior region of the stroma. Thus, when the irradiation step is resumed, the same problems persist.

In a method according to one embodiment of the invention, the barrier to diffusion formed by the anterior epithelium is reduced or eliminated. This step may be performed by any of the techniques discussed above in connection with the conventional methods.

The anterior surface of the cornea is then exposed to a first liquid with a first concentration of the catalyst. Typically, the first liquid includes a pharmaceutically acceptable solvent for the catalyst most typically an aqueous liquid such as balanced saline solution, and the catalyst is riboflavin 5′ phosphate sodium salt or another pharmaceutically acceptable form of riboflavin (simply referred to as ‘riboflavin’ throughout). Desirably, the first concentration is relatively high as, for example, perhaps as high as 0.5% riboflavin by weight. By starting with a high concentration, the method according to this embodiment exploits a large concentration gradient to rapidly push a significant amount of riboflavin to the stroma which diffuses according to the error function type solution to the Fick's second law. During this step, the boundary condition at the anterior surface of the cornea is set by the concentration of catalyst in the first liquid. The riboflavin concentration profiles 106, 108 and 110 within the stroma at three successive time points t1, t2, and t3 respectively during this period of high concentration exposure are shown in solid lines in FIG. 2. The concentration profiles formed in this stage of the method are similar to the concentration profiles formed in the conventional method. In these concentration profiles, the maximum riboflavin concentration in the stroma occurs at the anterior surface of the stroma, and the riboflavin concentration decreases monotonically in the posterior direction. If UV light were applied immediately following t3, most of the light would be blocked in the anterior of the stroma, severely impacting the effectiveness of the treatment. The riboflavin concentration profile at the end of this step (at t3) is referred to herein as the “first concentration profile”.

In the next stage of the method according to this embodiment, the anterior surface of the cornea is contacted with one or more additional liquids. Each of these additional liquids include a pharmaceutically acceptable solvent for the catalyst and has a catalyst concentration lower than the first catalyst concentration of the first liquid. Thus, the catalyst concentration of the liquid in contact with the anterior surface of the cornea is lowered to something much lower, typically to something close to the minimum concentration necessary for crosslinking. However, the riboflavin concentration of the additional liquid may be reduced all the way to zero. For example, pure saline solution may be used if desired (this may be done to facilitate a flush of the epithelial cells if they were left intact). The one or more additional liquids are or are contacted with the anterior surface of the cornea while the barrier function of the anterior epithelium remains disrupted or removed.

The riboflavin concentration in the liquid can be reduced from the first concentration to the lower concentration in an additional liquid either progressively or stepwise. While the eye is in contact with the additional liquid or liquids, riboflavin continues to diffuse. With the epithelial barrier compromised, the movement of the is governed by the concentration gradient. During this step, the boundary condition at the anterior surface is set by the catalyst concentration in the additional liquid. A low catalyst concentration in the additional liquid will tend to remove catalyst from the anterior portions of the stroma. This will reduce the concentration of catalyst in the anterior regions of the stroma. The catalyst concentration profiles 112, 114 and 116 formed at successive times t4, t5 and t6 during exposure to the additional liquids are shown diagrammatically in broken lines in FIG. 2. In each of these concentration profiles, the maximum concentration of riboflavin occurs at a location remote from the anterior surface of the stroma. Stated another way, the concentration of riboflavin in an anterior portion of the stroma is less than the maximum concentration of riboflavin in the stroma, and the maximum concentration of riboflavin in the stroma is disposed posterior to this anterior portion. Thus, over at least a portion of the concentration profile at and near the anterior surface of the stroma, the riboflavin concentration increases with depth from the anterior surface of the stroma. This effect, achieved by exposure to the one or more additional liquids, desirably is substantial. One measure of this effect is the distance or depth D_{MAX} from the anterior surface of the stroma to the location of the maximum concentration. D_{MAX} increases with time. Desirably, exposure to the one or more additional liquids continues until D_{MAX} is equal to at least 5%, more desirably at least 10% or more of the thickness of the corneal stroma. For example, in this embodiment, exposure to the second liquid ends at time t6. D_{MAX} at time t6 may be on the order of one half of the thickness of the stroma. Stated another way, this approach will tend to set up a ‘wave’ of riboflavin within the stroma, with a high concentration wave moving posteriorly, even as the peak of concentration within this wave is reduced.

Another measure of the effect achieved by exposure to the one or more additional liquids is the ratio between the catalyst concentration a thin layer of the stroma as, for example, a region 10-20 μm thick, centered at D_{MAX} and a layer of the stroma having same thickness beginning at the anterior surface of the stroma. Desirably, this ratio is at least 1.05:1, more desirably at least 1.25:1, and most desirably at least 1.5:1. This ratio is referred to herein as the D_{MAX} to anterior concentration ratio. In the embodiment, at time t6, the average concentration of riboflavin in an anterior portion of the stroma anterior to D_{MAX} is equal to or less than the average concentration in a posterior portion of the stroma posterior to D_{MAX}.

In a small region at the posterior extremity of the stroma, immediately adjacent the endothelium, the catalyst concentration may be reduced by diffusion of catalyst through the endothelium into the aqueous humor of the eye. This effect is not shown in FIG. 2. The concentration profile formed by exposure to the one or more additional liquids is referred to herein as the “second” concentration profile. The catalyst will continue to diffuse within the stroma after formation of the second concentration profile, with catalyst moving from the region of high concentration near D_{MAX} to the lower concentration regions, thus lowering the peak at D_{MAX} somewhat. Desirably, the concentration profile still retains the characteristics of the second concentration profile discussed above at the time the step of applying UV light, discussed below, begins.

In the next step of the method, UV light is applied to the cornea. The catalyst such as riboflavin is a very effective attenuator of UV light. The concentration of catalyst as a function of depth anterior to a target depth in the cornea greatly impacts the UV dose available at that target depth. The relatively low concentration of catalyst in the anterior region of the stroma allows more light to penetrate more deeply into the stroma, so that more cross-linking takes place deep within the stroma. This makes the treatment more effective. It is possible through careful manipulation of the catalyst concentrations in the first liquid and in the one or more additional liquids and the rate of change in concentration in the liquid, to construct a distribution of riboflavin that is effective for cross linking in the most anterior layers of the stroma but not so high as to stop the UV light penetration allowing cross linking in the deepest layers of the stroma as well. At the same time, the riboflavin concentration in the posterior stroma can be high enough to create an effective light block, preventing light from passing through the most posterior stroma and damaging the posterior endothelium.

The light-applying step may be interrupted, and additional catalyst may be applied to the cornea before resuming the light-applying step. This may be done one or more times. An individual reapplication may include contacting the cornea with a first liquid having a first concentration of the catalyst and then contacting the cornea with one or more additional liquids having concentrations of the catalyst lower than the first concentration. Stated another way, the steps used in the initial application of the catalyst, discussed above, may be repeated. The first liquid and the one or more additional liquids used in a repetition may be the same as those used in the initial application or may be different from those used in the initial application of the catalyst. Also, the duration of contact may be the same or different. When these steps are repeated, the resulting catalyst concentration profile will include some catalyst which remains from a previous application of catalyst. In some cases, the resulting catalyst concentration profile can include multiple peaks, i.e., multiple local maxima separated from one another by a region having lower concentration. For a concentration profile with multiple peaks, D_{MAX} should be taken as the distance from the anterior surface to the peak (local maximum) closest to the anterior surface.

One significant advantage which can be achieved by providing a concentration profile with low riboflavin concentration in the anterior stroma and high enough concentration in the posterior stroma is the ability to provide safe and effective crosslinking of thin corneas, such as those thinner than 400 microns. This is particularly significant in treatment of keratoconus (KC). Most conventional crosslinking protocols used in the clinic today for KC require that a patient's cornea be at least 400 microns thick to provide sufficient attenuation of UV light by the riboflavin-treated cornea. The more severe the KC, the more the patient can benefit from CXL. However, patients with severe KC have corneas thinner than 400 microns, so they are currently excluded from CXL treatment.

In a method according to a further embodiment of the invention, part the step of exposing the cornea to the one or more additional liquids occurs concomitantly with irradiation with UV light. As referred to in this disclosure, exposure to an additional liquids occurs “concomitantly with” irradiation either (i) where additional liquid exposure occurs simultaneously with irradiation, so that the UV light passes through the additional liquid to the cornea or (ii) where the irradiation is applied intermittently, and exposure to additional liquid occurs between the intermittent applications. After contact with a first additional liquid having a lower concentration of riboflavin than the first liquid, the cornea is irradiated with ultraviolet light. Concomitantly with irradiation, the epithelium is contacted by a further additional liquid which may have the same concentration of riboflavin as the first additional liquid or a different concentration. Thus, some further change in the concentration profile will occur during the irradiation step. Where the further additional liquid is applied simultaneously with irradiation, the further additional liquid preferably has zero riboflavin concentration when applied to the cornea, and desirably is applied in a continuous or intermittent flow over the cornea, so that liquid which has extracted riboflavin from the cornea is replaced by fresh liquid. This keeps the riboflavin concentration in the liquid overlying the cornea at or near zero, so that the applied UV light is not absorbed by riboflavin in the liquid.

In some embodiments, the step of irradiating the cornea includes varying one or more characteristics of the applied light, such as the power of the light in the course of the irradiation step. The pattern of variation in the applied light may be selected so that the characteristics of the applied light at each time, in conjunction with the concentration profile at that time, produces the desired pattern of crosslinking within the stroma. For example, the variation in the concentration profile with time can be predicted based on the riboflavin concentrations of the liquids to be used, the timing of their application and the thickness of the cornea. Based on the predicted concentration profile, the absorption of light by the cornea and the crosslinking rate at each point within the cornea for a given applied light power can be modeled. The modeling also may take account of predicted losses of riboflavin due to factors such as photobleaching. In other variants, the actual concentration profile can be measured continuously or intermittently during the irradiation step and the applied light may be adjusted based at least in part on the measured concentration profile.

In another embodiment, a barrier to diffusion of riboflavin is restored at the anterior surface of the cornea after contact with the one or more additional liquids but before the end of the irradiation step so that the barrier is present during at least part of the irradiation step. Ideally, the barrier is restored before commencement of the irradiation step and is present throughout the irradiation step. Where the anterior epithelium remains in place and the barrier function of the epithelium is reduced or eliminated by treating the epithelium with a drug, the barrier can be restored by reversing the effect of the drug, for example by through passage of time and dissipation of the effect, by applying an antidote to the drug, by breaking the drug down with UV, by washing of the drug out of the epithelium or by combinations of these processes. In a further variant, the barrier is restored by contacting the anterior surface of the cornea with a barrier material which has low or zero solubility for riboflavin and which is transparent to UV light. One such material is perfluorocarbon. As disclosed in U.S. Pat. No. 10,010,449, the disclosure of which is incorporated by reference herein, a liquid perfluorocarbon may be maintained in contact with the anterior surface of the cornea during UV irradiation by a structure resembling a contact lens. The perfluorocarbon may contain dissolved oxygen to maintain oxygen tension in the cornea during crosslinking. Because riboflavin (ions in aqueous solution from riboflavin 5′ phosphate sodium) is essentially insoluble in perfluorocarbon, a liquid perfluorocarbon will act as a barrier material. In a further variant, a coating of perfluorocarbon may be applied to the anterior surface of the cornea so that the coating remains on the cornea without a retaining structure.

In one variant the liquid perfluorocarbon barrier fluid could be introduced continuously or recirculated using an active or gravity pumping system, continuously supplying the cornea with a constant O₂ concentration at its anterior surface, the recirculation technique requiring reoxygenation prior to re-introduction.

This restored barrier prevents the riboflavin from diffusing back out into the space anterior to the cornea. In this manner, the riboflavin would be effectively trapped in the cornea, with any reduction in the total amount of catalyst in the stroma during the UV treatment due only to diffusion through the endothelium into the anterior chamber of the eye, or photodegradation of the riboflavin. Desirably, the riboflavin loading scheme prior to irradiation instills enough riboflavin to account for these loss mechanisms and maintain the riboflavin level above the minimum necessary for crosslinking without the need to reapply riboflavin during the therapy.

Stated another way, by starting with a bolus of the a liquid with a high riboflavin concentration, then reducing the concentration in the anterior stroma in a controlled manner during a presoak before irradiation, the gradient in the stroma can be engineered so that the concentration at depth is greater than that in the anterior section, allowing the entire stroma to be crosslinked without risking UV damage to the endothelium.

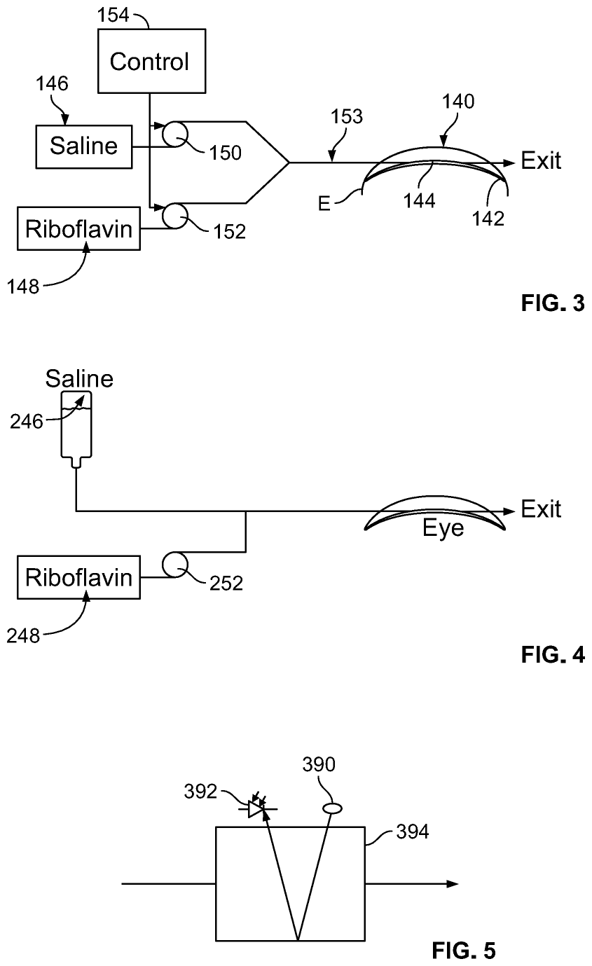

One apparatus suitable for performing a method as discussed above is depicted in FIG. 3. The apparatus includes a reservoir 140 which may be generally in the form of a contact lens. The reservoir has an inner surface 142 arranged to rest on an anterior surface of the eye E while leaving a space 144 between the anterior surface of the cornea and the apparatus. For example, the reservoir may be generally in the form of a scleral contact lens adapted to rest on the anterior surface of the sclera. The reservoir desirably has an inlet port which communicates with the space when the reservoir is in place on the eye, and may also have an outlet port communicating with the space. The apparatus may include a source 146 of a liquid such as saline solution or other physiologically acceptable liquid, and a source 148 of a concentrated riboflavin solution. As depicted in FIG. 3, the apparatus includes a pump 150 connected to source 146 and another pump 152 connected to the source 148. The outflow connections of the pumps are linked to a fluid supply conduit 153, which in turn is connected to the space 144 via the inlet port of the reservoir. In this embodiment, the apparatus includes a control circuit arranged to control operation of the pumps 150 and 152. Where two liquids are supplied simultaneously, they blend with one another to form a blended liquid which is supplied to the space 144. Varying the proportion of different liquids varies the concentration of riboflavin in the liquid supplied to the space. In operation, the liquid supplied by the two pumps mixes in the supply conduit, passes into the space through the inlet port, and out of the space through the exit port.

In a variant, the exit port may be omitted and the fluid may pass out of the space by leakage between the interior surface of the reservoir and the eye. In a further variant, the reservoir may be a ring-like structure defining a space open to the atmosphere, and the patient may be positioned so that the open side of the reservoir faces upwardly. In this embodiment, the fluid can pass out of the space over the top side of the ring, or through a drain port.

The reservoir may be arranged so that ultraviolet light may be applied to the cornea while the reservoir remains in place on the eye. For example, the reservoir may be a structure as disclosed in U.S. Pat. No. 9,907,698 the disclosure of which is hereby incorporated by reference herein. These structures include elements such as optical fibers for supplying ultraviolet light from an external source or light-emitting diodes for generating ultraviolet light, and optical elements for directing the light into the cornea. In another arrangement, the reservoir may be transparent to ultraviolet light and light may be directed into the cornea from a remote source while the reservoir remains in place.

Other fluid-supplying structures may be substituted for one or both of the pumps shown in FIG. 3. For example, as shown in FIG. 4, the source 246 of saline includes a bottle or bag similar to those used in intravenous therapy for supplying fluid by gravity feed, whereas the source 248 is linked to a pump 252 which is controlled by the control system (not shown).

In a further variant, multiple sources of pre-mixed liquids at different riboflavin concentrations can be used. These sources can be connected to the supply conduit in succession, either manually or automatically, so as to provide a desired succession of riboflavin concentrations.

The apparatus may include a device adapted to measure the riboflavin concentration in the liquid supplied to the space, and the control system may be responsive to the measured concentration so as to provide the desired variation in concentration with time. For example, as shown in FIG. 5, a photometer includes a source 390 of ultraviolet light or light at another wavelength or a spectrum of wavelengths absorbed by riboflavin. The light source may be a light emitting diode, laser or lamp. The photometer also includes a photodiode 392. The light source is arranged to direct low intensity light through the liquid in a chamber 394. Chamber 394 is connected to the flow path of the liquid as, for example, in the supply conduit 153 of the apparatus shown in FIG. 1. The chamber is arranged so that light from source 390 which has passed through the liquid impinges on the photodiode. The photodiode generates a signal which varies with the concentration of riboflavin in the liquid. The chamber may be included in the inlet conduit, included in a bypass line connected to the inlet conduit, or may be the space between the reservoir and the eye.

In another embodiment, the photodiode is sensitive to the fluorescence spectrum of the riboflavin and the wavelength of the low intensity light source is selected to excite fluorescence, the intensity of which is then used to calculate the concentration of the riboflavin.

In yet another variant, a photometer similar to one of the two described above is used to monitor the riboflavin concentration in the cornea itself, and the control system is responsive to the measured concentration in the cornea.

In yet another embodiment, the photosensor system (either absorbance or fluorescence) may connected to the outlet port to monitor riboflavin leaving the cornea.

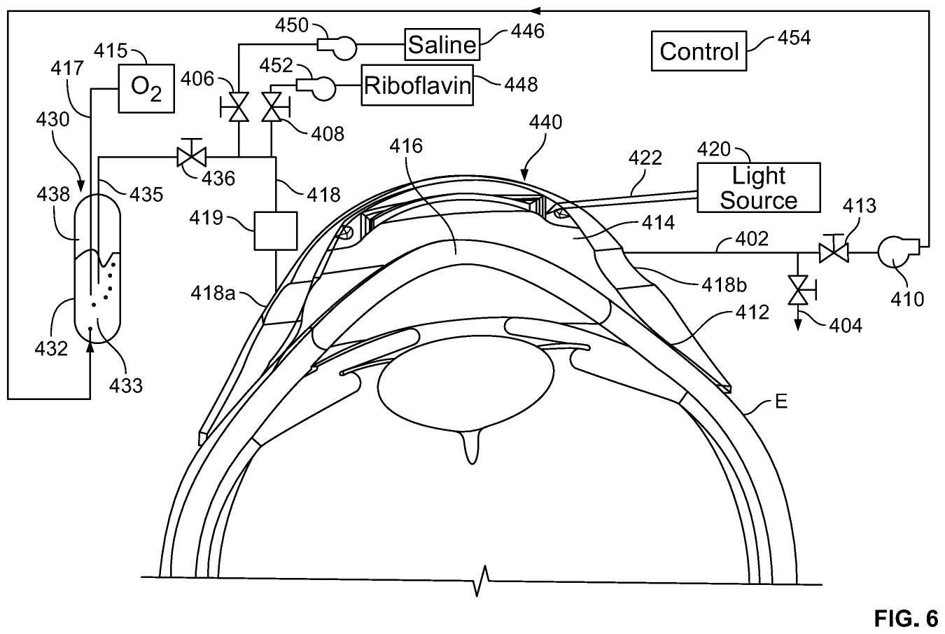

The apparatus depicted in FIG. 6 includes a reservoir including a structure 440 having a shape and size similar to that of a conventional scleral contact lens. The structure includes an interior surface 412 with a shape adapted to contact the sclera of the eye E and to leave a space 414 between the cornea 416 and the interior surface. The structure has an inlet port 418a and an outlet port 418b. In this embodiment, the structure includes optical elements adapted to direct light such as ultraviolet light from a light source 420 such as a laser into the cornea 416 while the structure is in place on the cornea. The optical elements may be as described in U.S. Pat. No. 9,907,698. For example, the optical elements may include one or more optical fibers 422 in optical communication with the light source and may also include reflective elements (not shown) for routing the light from the fibers into the cornea. The optical elements may include optically scattering elements such as diffuse reflecting surfaces, scattering transmissive elements and the like, as well as a wall defining an aperture (not shown) for limiting light application to a desired region of the cornea. In other embodiments, the elements for directing the light into the cornea may include light-emitting elements such as light-emitting diodes. In still other embodiments, structure 440 may be formed partially or entirely from a material which is transparent to light at the wavelength of the light used for corneal crosslinking, so that light from an external source can be directed through the structure into the cornea.

The apparatus of FIG. 6 further includes a supply conduit 453 connected to inlet port 418a and an outlet conduit 402 connected to outlet port 418b. A drain valve 404 is connected to the outlet conduit 404. Alternatively, drain valve 404 may be connected directly to space 416. The apparatus in this embodiment further includes a source of a liquid containing catalyst such as riboflavin 448 linked to a pump 452; a source of a solvent such as saline 446 and a pump 450 linked to source 446. These elements are connected to supply conduit 418 through valves 406 and 408. The apparatus of FIG. 6 includes a further liquid source 430 containing a barrier liquid 433 such as a perfluorocarbon. In this embodiment, the supply source is in the form of a closed tank 432 holding the liquid at the bottom. A dip tube 435 extends into the tank to below the liquid level. The dip tube is connected to the supply conduit 418 through a valve 436. Desirably, the liquid in tank 432 is under superatmospheric pressure. A recirculation pump 410 is connected to outlet conduit 402 through a further valve 413. A source 415 of oxygen or a gas mixture containing oxygen such as air is connected to a bubbler tube 417 extending into tank 432 below the level of the barrier liquid, so that the barrier liquid 433 in the tank is maintained saturated with oxygen. A control system 454 is operable to command the valves and pumps to perform the operations discussed below. Optionally, the controller receives input from a photometer 419 in the supply conduit as shown or in the outlet conduit 402 to monitor riboflavin concentration of liquid passing into or out of space 414.

In operation, with valves 436 and 413 shut, pumps 452 valve 408 can be actuated to feed a first liquid from source 448 through valve 408 and into space 414. Optionally, some solvent from source 446 may be introduced through pump 450 and valve 406 so that the solvent forms part of the first liquid. Drain valve 404 is maintained open until the first liquid having the desired catalyst concentration has purged space 414. The cornea 416 is contact by the first liquid for a desired time to form a first catalyst concentration profile. Then, one or more additional liquids with lower concentration of catalyst are supplied to space 414 from source 446 and optionally source 448 with the associated pumps and valves so as to maintain a desired catalyst concentration in these additional liquids. Drain valve 404 may remain open so that the additional liquids flow continually through space 414. After the cornea has been contacted with the additional liquid or liquid for the desired time to form the second concentration profile, valves 406 and 408 are closed and valve 436 is opened so that the inlet conduit space 414 and the outlet conduit are purged of the catalyst-containing liquids and filled with the barrier liquid through drain valve 404. The drain valve is closed and recirculation pump 410 is operated to return barrier liquid from space 414 to tank 432 of source 430. The barrier liquid circulates through the tank where it is reoxygenated, and then returns to the space 414 so as to keep keeping the space between the reservoir and the eye at least partially filled with the barrier liquid, and desirably completely filled. The light source 420 is actuated to direct light, typically UV light, into the cornea so as to bring about the desired crosslinking of collagen in the stroma. Filling the space with a barrier liquid having very low or zero solubility for the catalyst during the irradiation step assures that the space remains substantially free of catalyst so that the light applied during irradiation is not absorbed by catalyst. The barrier liquid helps to maintain a controlled high oxygen content in the corneal stroma.

The features of the apparatus discussed above with respect to FIG. 6 can be varied. For example, oxygen concentration of the barrier liquid can be maintained by measures other than the bubbler tube 417 as, for example, by an oxygenation device separate from reservoir 430. Also, although the apparatus of FIG. 6 is shown and described herein in conjunction with the use of first and second liquids as used in other aspects of the invention, it the recirculation of an oxygen-containing liquid such as a perfluorocarbon as shown and discussed with reference to FIG. 6, this arrangement is beneficial regardless of the catalyst concentration profile used. Thus, the apparatus may omit the source 446 used to form the one or more additional liquids and the associated components so that the catalyst profile during irradiation is a conventional profile. Indeed, the apparatus may omit catalyst source 448 and the associated components, and may be used to apply an oxygenated liquid after catalyst has been introduced into the stroma by conventional means to form a conventional catalyst concentration profile.

In those methods where a barrier is not used and one or more additional liquids which are solvents for riboflavin are applied during the irradiation step, the one or more additional liquids desirably are fed through the space in a continuous or intermittent flow during irradiation. This maintains the concentration of riboflavin in the liquid overlying the cornea at a very low level, desirably a constant level, and thus limits absorption of the applied light by riboflavin present in the liquid.

In the apparatus discussed above, a single reservoir is used for all of the steps. In other embodiments, two or more reservoirs are used during different steps of the method. For example, a first reservoir can be used to apply the liquids so as to form the first and second concentration profile, and a second reservoir may be used during irradiation. The first reservoir need not be arranged for applying light to the eye. Either reservoir can be used to apply a barrier such as perfluorocarbon to the eye. For example, the second reservoir can be filled with perfluorocarbon liquid as it is applied to the eye so that the eye displaces the perfluorocarbon as the lens is placed onto the eye. This procedure can be similar to the procedure used to fill a conventional scleral contact lens with saline before applying it to the eye. In a further variant, a barrier liquid can be applied to the eye, as with an eye dropper, before applying the second reservoir. In yet another variant, a reservoir which is in place can be filled with air or another gas, so that the anterior surface of the cornea is in contact with the gas. The gas serves as a barrier to diffusion of riboflavin through the anterior surface.

In other arrangements, the reservoir is removed before irradiating the cornea. For example, the reservoir used to form the second concentration profile may be removed from the eye after formation of the second concentration profile, and the cornea may be irradiated with light directed into the cornea from a source remote from the eye. In still other arrangements, the liquids including the first liquid, additional liquids and barrier, if used, may be applied to the eye without use of reservoirs, as by continually directing a slow stream or droplets into the eye.

The methods and apparatus discussed above also can be applied where corneal crosslinking catalysts other than riboflavin are employed. Likewise, light other than ultraviolet light can be used.

1. A method of corneal crosslinking comprising the steps of:
(a) maintaining the anterior surface of the cornea of an eye of a living subject in contact with a first liquid having a first concentration of a crosslinking catalyst dissolved therein so that the catalyst penetrates into the cornea and forms a first concentration profile within the stroma of the cornea; then
(b) after step (a) maintaining the anterior surface of the cornea in contact with one or more additional liquids having concentrations of the catalyst lower than the first concentration so that catalyst within the cornea forms a second concentration profile within the stroma different from the first concentration profile; and
(c) irradiating the cornea with light so that the light, in conjunction with the catalyst, causes crosslinking of collagen in the stroma, wherein step (b) is performed before step (c), concomitantly with step (c), or both before and concomitantly with step (c).

2. A method as claimed in claim 1 wherein the second concentration profile formed in step (b) has a lower concentration of the catalyst in an anterior portion of stroma of the cornea than the first concentration profile.

3. A method as claimed in claim 2 wherein, in the second concentration profile, a concentration of the catalyst in an anterior portion of the stroma is less than or equal to a maximum concentration of the catalyst in the stroma and the maximum concentration of the catalyst in the stroma is disposed at a location posterior to the anterior portion of the stroma.

4. A method as claimed in claim 3 wherein, in the second concentration profile, the maximum concentration of the catalyst in the stroma occurs at a depth from the anterior surface of the corneal stroma equal to at least 5 percent of the thickness of the corneal stroma.

5. A method as claimed in any of claim 1 further comprising treating or removing the anterior epithelium of the cornea so as to reduce or eliminate a barrier to diffusion of the catalyst formed by the anterior epithelium.

6. A method as claimed in claim 5 wherein step (b) is performed before step (c).

7. A method as claimed in claim 6, the method further comprising the step of restoring a barrier to diffusion of the catalyst at the anterior surface of the cornea before step (c).

8. A method as claimed in claim 7 wherein the step of restoring a barrier includes contacting the anterior surface of the cornea with a barrier material, the catalyst being substantially insoluble in the barrier material.

9. A method as claimed in claim 7 wherein the barrier material is a liquid.

10. A method as claimed in claim 9 wherein the barrier material is an oxygen-bearing liquid.

11. A method as claimed in claim 10 wherein the barrier material is a perfluorocarbon having oxygen dissolved therein.

12. A method as claimed in claim 7 wherein the step of treating or removing the anterior epithelium is performed by treating the epithelium with a drug and the step of restoring a barrier is performed by at least partially restoring the barrier to diffusion of the catalyst formed by the anterior epithelium.

13. A method as claimed in claim 5 wherein at least a part of step (b) is performed concomitantly with step (c).

14. A method as claimed in claim 13 further comprising predicting or measuring the second concentration profile at different times during step (c), wherein step (c) includes varying a characteristic of the light based at least in part on the predicted or measured second concentration profiles.

15. A method as claimed in claim 1 wherein step (a) includes positioning a reservoir on the eye so that the reservoir defines a space aligned with the cornea and introducing the first liquid into the space.

16. A method as claimed in claim 15 wherein step (b) includes introducing a first one of the one or more additional liquids into the space so that the first one of the one or more additional liquids displaces the first liquid from the space.

17. A method as claimed in any claim 1 wherein the catalyst is riboflavin.

18. Apparatus for treating the eye of a living subject with a catalyst for corneal crosslinking comprising:
(a) at least one reservoir adapted to rest on the eye of the subject and to define a space in alignment with the cornea of the eye;
(b) a plurality of liquid sources holding a plurality of liquids having different concentrations of riboflavin, at least one of the concentrations of riboflavin being greater than zero; and
(c) means for supplying one or more of the liquids to the space so as to provide liquid in the space and vary a concentration of riboflavin of liquid in the space with time and thereby contact the cornea with a succession of liquids including a first liquid having a first concentration of riboflavin and one or more additional liquids having concentrations of riboflavin lower than the first concentration.

19. Apparatus as claimed in claim 18 wherein the means for supplying includes means for blending liquids from a plurality of sources and supplying the blended liquid to the space.

20. Apparatus as claimed in claim 19 wherein the means for supplying is operative to vary the proportions of liquids from different sources included in the blended liquid so as to vary a concentration of riboflavin in the blended liquid.

21. Apparatus as claimed in claim 18 wherein the means for supplying is operative to pass the first liquid and the one or more additional liquids to the space so that such liquids flow through the space and out of the space while contacting the cornea.

22. Apparatus as claimed in claim 18 wherein the at least one reservoir includes a reservoir which is adapted to transmit light to the cornea of the eye to activate the catalyst.

23. Apparatus for treating the eye of a living subject with a catalyst for corneal crosslinking comprising:
(a) means for maintaining the anterior surface of the cornea of an eye of a living subject in contact with a first liquid having a first concentration of a crosslinking catalyst dissolved therein so that the catalyst penetrates into the cornea and forms a first concentration profile within the stroma of the cornea;
(b) means for maintaining the anterior surface of the cornea in contact with one or more additional liquids having concentrations of the catalyst lower than the first concentration after the first concentration profile has been formed so that catalyst within the cornea forms a second concentration profile within the stroma different from the first concentration profile; and
(c) means for irradiating the cornea with light so that the light, in conjunction with the catalyst, causes crosslinking of collagen in the stroma.

24. Apparatus as claimed in claim 23 wherein the means for maintaining the anterior surface of the cornea in contact with the more additional liquids is operative to maintain the anterior surface of the cornea in contact with the one or more additional liquids so that, in the second concentration profile, a concentration of the catalyst in an anterior portion of the stroma is less than a maximum concentration of the catalyst in the stroma and the maximum concentration of the catalyst in the stroma is disposed at a location posterior to the anterior portion of the stroma.

25. Apparatus as claimed in claim 24 wherein the means for maintaining the anterior surface of the cornea in contact with the more additional liquids is operative to maintain the anterior surface of the cornea in contact with the one or more additional liquids so that, in the second concentration profile, the maximum concentration of the catalyst in the stroma occurs at a depth from the anterior surface of the corneal stroma equal to at least 5 percent of the thickness of the corneal stroma.

26. Apparatus as claimed in claim 23 wherein the means for irradiating is operative to begin irradiation of the cornea after the means for maintaining the anterior surface of the cornea in contact with the more additional liquids has begun such contact.

27. Apparatus as claimed in claim 26 wherein the means for irradiating is operative to begin irradiation of the cornea after the means for maintaining the anterior surface of the cornea in contact with the more additional liquids has terminated such contact.

28. A method as claimed claim 23 further comprising means for restoring a barrier to diffusion of the catalyst at the anterior surface of the cornea.

29. A method as claimed in claim 28 wherein the means for restoring a barrier includes means for contacting the anterior surface of the cornea with a barrier material, the catalyst being substantially insoluble in the barrier material.

30. A method as claimed in claim 29 wherein the barrier material is a liquid and the means for restoring a barrier includes means for supplying the liquid barrier material with oxygen dissolved therein.
